(19) United States
(12) Patent Application Publication
(10) Pub. No.: US 2017/0354670 A1
(43) Pub. Date: Dec. 14, 2017

(54) PRODUCT COMPRISING GLUCOSAMINE FOR EXTERNAL APPLICATION

(71) Applicant: Vital Medicine, LLC, Morristown, NJ (US)

(72) Inventors: David H. Schwartz, Warren, NJ (US); Steven M. Weisman, Morristown, NJ (US)

(73) Assignee: Vital Medicine, LLC, Morristown, NJ (US)

(21) Appl. No.: 15/176,483

(22) Filed: Jun. 8, 2016

(51) Int. Cl. A61K 31/7008; A61K 9/70; A61M 35/00; A61K 45/06; A61K 9/00; A61F 13/40

(52) U.S. Cl. CPC A61K 31/7008; A61K 9/0014; A61M 35/006; A61M 35/00; A61K 45/06; A61K 9/7023; A61M 2210/08

(57) ABSTRACT

The present disclosure relates to, inter alia, a topical fluid application apparatus comprising a solution of glucosamine or a pharmaceutically acceptable salt thereof; an article of manufacture that is damp or impregnated with a solution of glucosamine or a pharmaceutically acceptable salt thereof; a kit comprising the topical fluid application apparatus and/or the article of manufacture disclosed; and methods of using the topical fluid application apparatus, the article of manufacture, and/or the kit.

### TECHNICAL FIELD

This invention relates to the field of relief of pain or discomfort.

### BACKGROUND

The external body suffers from pain or discomfort, due to many reasons. For example, joint conditions affect millions of individuals and can cause great pain or discomfort.

### SUMMARY

This disclosure provides a number of products and methods to relieve pain or discomfort in a subject, regardless of the cause.

In some aspects, this disclosure provides a topical fluid application apparatus. The topical fluid application apparatus comprises glucosamine or a pharmaceutically acceptable salt thereof in a solution. In certain embodiments, the topical fluid application apparatus is a flexible plastic form that fits over the hand or foot of a subject. In certain further embodiments, the flexible plastic form is a bootie. In yet other embodiments, the flexible plastic form is a glove.

In other aspects, this disclosure provides an article of manufacture. The article of manufacture is damp or impregnated with a solution comprising glucosamine or a pharmaceutically acceptable salt thereof. In some embodiments, the article of manufacture is a pliable sponge, a moist towelette, or a pad.

In other aspects, this disclosure provides a kit comprising:

a flexible plastic form that fits over the hand or foot, a pliable sponge, or both; a container of a solution comprising glucosamine or a pharmaceutically acceptable salt thereof; and instruction to add the solution to the plastic form or dampen the sponge, or both, just prior to use.

In other aspects, this disclosure provides a method of reducing pain or discomfort in a subject comprising immersing an area of the subject's body in pain in a topical fluid application apparatus disclosed herein, treating the area of the subject's body in pain with an article of manufacture disclosed herein, or using the kit disclosed herein to treat the subject.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1 illustrates an embodiment of a bootie of this disclosure.

FIG. 2 illustrates an embodiment of a glove of this disclosure.

FIG. 3 illustrates an embodiment of a sponge of this disclosure.

FIG. 4 illustrates an embodiment of a box of this disclosure.

FIG. 5 and FIG. 6 shows study data of Example 1.

FIG. 7 shows a graph plotting pain vs. time.

FIG. 8 shows result of T test of Example 1.

FIG. 9 shows ANOVA data of Example 1.

FIG. 10 shows ANOVA analysis of Example 1.

FIG. 11 shows a graph plotting “desire to purchase.”

### DETAILED DESCRIPTION

As used herein, the word “a” or “plurality” before a noun represents one or more of the particular noun. For example, the phrase “a subject” represents “one or more subjects.”

As used herein, the terms “subject” and “patient” are used interchangeably. A patient or a subject can be a human patient or a human subject. A subject or a patient can be an animal, for example, a dog, a cat, a monkey, etc. The animal can be a domestic animal (a pet), such as a dog, a cat, etc.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

This disclosure provides a topical fluid application apparatus. The topical fluid application apparatus comprises glucosamine or a pharmaceutically acceptable salt thereof in a solution. In certain embodiments, the topical fluid application apparatus is a flexible plastic form that fits over the hand or foot of a subject. In certain further embodiments, the flexible plastic form is a plastic bootie. In yet other embodiments, the flexible plastic form is a glove.

In other aspects, this disclosure provides an article of manufacture. The article of manufacture is damp or impregnated with a solution comprising glucosamine or a pharmaceutically acceptable salt thereof. In some embodiments, the article of manufacture is a pliable sponge, a moist towelette, or a pad.

In other aspects, this disclosure provides a kit comprising:

a flexible plastic form that fits over the hand or foot, a pliable sponge, or both; a container of a solution comprising glucosamine or a pharmaceutically acceptable salt thereof; and instruction to add the solution to the plastic form or dampen the sponge, or both, just prior to use.

In other aspects, this disclosure provides a method of reducing pain in a subject comprising applying to an area of the subject's body in pain a topical fluid application apparatus disclosed herein, treating the area of the subject's body in pain with an article of manufacture disclosed herein, or using the kit disclosed herein to treat the subject.

As used herein, “a product (or products) disclosed herein” refers to any composition, topical fluid application apparatus, article of manufacture, or kit comprising glucosamine or a pharmaceutically acceptable salt thereof and adapted for provision of glucosamine or a pharmaceutically acceptable salt thereof to a subject.

Some exemplary embodiments of the invention are shown in Table 1.

TABLE 1

| | |
|---|---|
| Apparatus | foot-shaped (bootie); |
| Shape | hand-shaped (glove); |
| | pliable sponge, moist towelette, or pad |
| | box or container |
| Action on body | relieves pain, discomfort, aches, stiffness, and stress |
| (method of use) | relieves tired achy feet, hands, and joints |
| | provides soothing comfort |
| | decreases symptoms of arthritis |
| | provides analgesia and anti-inflammatory properties |
| Delivery | article containing with glucosamine solution |
| | article containing dry glucosamine (add water) |
| | article impregnated with dry glucosamine (occlusion - |
| | add sweat) |
| Actives | glucosamine |
| | N-aacetylglucosmaine |
| | glucosamine potassium sulfate and glucosamine |
| | sulfate |
| | glucosamine hydrochloride or glucosamine potassium |
| | hydrochloride |
| | any glucosamine salt |
| | any combination of the above |
| Modification | permeation or penetration enhancer |
| of glucosamine | chondroitin sulfate or methylsolfonylmethane (MSM) |
| action | counter-irritants such as menthol, camphor, capsaicin |
| | Epsom salts |
| | Analgesics, anti-inflammatory agents |
| | other counter-irritants |
| | other drugs or cosmetic ingredients |
| Subjects | humans |
| | animals (e.g., companion animals and veterinary uses) |

Topical Fluid Application Apparatus

In some embodiments, a topical fluid application apparatus is provided. The topical fluid application apparatus comprises glucosamine or a pharmaceutically acceptable salt thereof in a solution, wherein said solution is either added externally or results from a subject's sweat. A topical fluid application apparatus is used for application of an external part of the body to a liquid solution. In certain embodiments, the topical fluid application apparatus is a flexible plastic form that fits over the hand or foot of a subject. In certain further embodiments, the flexible plastic form is a bootie. The bootie can contain (1) solution; (2) a pad; or (3) dry or impregnated powder that would be solubilized by water or body fluids (e.g., sweat). In yet other embodiments, the flexible plastic form is a glove. The glove can contain (1) solution; (2) a pad; or (3) dry or impregnated powder that would be solubilized by water or body fluids (e.g., sweat).

Other suitable topical fluid application apparatus including other suitable flexible plastic forms are contemplated, ones that can hold a solution comprising glucosamine or a salt thereof and be fitted on a part of a body of a subject. The solution can be one that is added or it can be induced by occlusion of skin (i.e., sweat) of the subject. Any suitable kind of plastic may be used. The topical fluid application apparatus may be made with other suitable materials (not plastic).

In certain embodiments, the topical fluid application apparatus is a box that resembles a shoe box and is a container, such as a plastic container, that fits over the foot of a subject. The box holds solution (the solution can be one that is added or it can be induced by occlusion of skin (i.e., sweat) of the subject). The box may be made of plastic (any suitable plastic) or other suitable materials. The box may be of any suitable size; including a size that will allow a foot to fit into it.

The volume of the solution can be any suitable volume, including very small volume.

In some embodiments, a part of a subject's body, most of the subject's body, or even the entire body, can be immersed in a topical fluid application apparatus, in which the glucosamine or a pharmaceutically acceptable salt thereof (and optionally additional agents) is delivered. The part of the body that is immersed in a topical fluid application apparatus may be the part of the body in need of treatment (for example, in pain).

In some embodiments, the subject's body is partially or completely immersed in the topical fluid application apparatus such that the blood plasma level or tissue level of the glucosamine or a pharmaceutically acceptable salt thereof is raised in the subject.

The temperature of the solution in the topical fluid application apparatus can be any suitable temperature, including room temperature. Any suitable amount of glucosamine or a pharmaceutically acceptable salt thereof is contemplated. In some embodiments, the quantity of glucosamine can be determined based on the BMI, size, height, age, etc. of the subject. A topical fluid application apparatus described herein (i.e., a soak) can be prepared according to any and all suitable parameters. See U.S. Pat. No. 9,012,430.

Article of Manufacture

In some embodiments, an article of manufacture is provided. The article of manufacture is dampened or impregnated with a solution comprising glucosamine or a pharmaceutically acceptable salt thereof. The solution can be one that is added or it can be induced by occlusion of skin (i.e., sweat) of the subject.

In some embodiments, the article of manufacture is a pliable sponge, a moist towelette, or a pad. In certain embodiments, the article of manufacture is a wrap. Any suitable article of manufacture is contemplated, one that can be dampened or impregnated with solution comprising glucosamine or a pharmaceutically acceptable salt thereof.

In certain embodiments, the pliable sponge is covered by a plastic cover. The plastic cover prevents the liquid from dampening items such as clothes or bedding. Any suitable plastic cover is contemplated. The pliable sponge can be any suitable size, including the size of a bandaid (bandaid of any size, including a large bandaid). The pliable sponge may be attached to the skin with an adhesive. The sponge may optionally have an external plastic cover so as not to leak the solution. The sponge can be any size so as to accommodate a particular affected body part or site. The sponge can also take a custom shape to fit a given body part (e.g., shaped like a foot, a hand, a shoulder, or a knee).

The moist towelette can be individually wrapped and can be of any suitable size.

The pad can be packaged together in a container and can be of any suitable size.

The wrap can be used to wrap around a part of a subject's body. The wrap can be any suitable size. The wrap can be in the form of an adhesive.

The article of manufacture can be of any suitable size and made by any suitable material.

Each article of manufacture is damp or impregnated with any suitable amount of a solution comprising glucosamine or a pharmaceutically acceptable salt thereof, thus allowing applying the article of manufacture on a part of the body of a subject, a part in need of such application, such as, for example, a part of the subject's body that is in pain.

Kit

In some embodiments, a kit is provided. The kit comprises:

a flexible plastic form disclosed herein that fits over the hand or foot, and/or an article of manufacture disclosed herein; a container of a solution comprising glucosamine or a pharmaceutically acceptable salt thereof;
and instruction to add the solution to the plastic form and/or dampen the article of manufacture, just prior to use.

In some embodiments, a kit is provided. The kit comprises:

a flexible plastic form disclosed herein that fits over the hand or foot, and/or an article of manufacture disclosed herein; and instruction to induce formation of solution in the plastic form and/or the article of manufacture, just prior to use, by occluding the skin of the subject, thus producing sweat.

Other kits comprising other product(s) disclosed herein and instruction for use are also comtemplated.

Glucosamine

Glucosamine (molecular formula C₆H₁₃N0₅) is a naturally occurring monosaccharide amino sugar, also known as (3R,4R,5S)-3-Amino-6-(hydroxymethyl)oxane-2,4,5-triol and 2-Amino-2-deoxy-glucose chitosamine. Glucosamine is available in several suitable chemical forms. The glucosamine or a pharmaceutically acceptable salt thereof may be glucosamine and/or N-acetylglucosamine. Many salts of glucosamine are suitable. In some embodiments, the glucosamine or a pharmaceutically acceptable salt thereof is glucosamine, N-acetylglucosamine, glucosamine sulfate, glucosamine hydrochloride, glucosamine potassium sulfate, glucosamine hydrochloride, glucosamine potassium hydrochloride, glucosamine sulfate and combinations thereof.

A product disclosed herein (which comprises glucosamine or a pharmaceutically acceptable salt thereof) can be administered in combination with one or more additional agents. Any suitable or additional agent is contemplated.

In some embodiments, a product disclosed herein (which comprises glucosamine or a pharmaceutically acceptable salt thereof) further comprises chondroitin (e.g., chondroitin sulfate), methylsulfonylmethane, hyaluronic acid, omega 3 fatty acids (e.g., eicosapentaenoic acid, docosahexaenoic acid, α-linolenic acid, etc.), vitamin D, vitamin C, quercetin, avocado soybean unsaponifiables (ASU), antioxidant berry extracts (e.g., blueberries, acai berry, maqui berry, chokeberry, etc.), COX inhibitors (e.g., non-steroidal anti-inflammatory agents), turmeric (Curcuma/onga), bromelain, and combinations thereof.

Chondroitin sulfate is a sulfated glycosaminoglycan (GAG) composed of an unbranched polysaccharide chain of variable length containing two alternating monosaccharides: D-glucuronic acid (GlcA) and N-acetyl-D-galactosamine (GalNAc). In some cases, a chondroitin chain is composed of over 100 individual sugars, each of which can be sulfated in variable positions and quantities. Chondroitin sulfate is an important structural component of cartilage and provides much of its resistance to compression.

In some embodiments, a product disclosed herein (which comprises glucosamine or a pharmaceutically acceptable salt thereof) further comprises methylsulfonylmethane. Methylsulfonylmethane (molecular formula C₂H₆0₂S; structural formula (CH₃)₂S0₂) is an organosulfur compound. It is also known by several other names including MSM, DMS0₂, methyl sulfone, and dimethyl sulfone.

In further embodiments, the product disclosed herein comprises not only glucosamine or a pharmaceutically acceptable salt thereof, but also chondroitin and methylsulfonylmethane.

In some embodiments, the product disclosed herein (which comprises glucosamine or a pharmaceutically acceptable salt thereof) further comprises hyaluronic acid. Hyaluronic acid is an anionic, nonsulfated glycosaminoglycan (GAG), also known as hyaluronan. Hyaluronic acid is a polymer of disaccharides, themselves composed of D-glucuronic acid and N-acetyl-D-glucosamine, linked via alternating β-1,4 and β-1,3 glycosidic bonds.

In some embodiments, the product disclosed herein (which comprises glucosamine or a pharmaceutically acceptable salt thereof) further comprises a counterirritant, such as, for example, menthol, camphor, capsaicin, eucalyptol, methyl salicylate, and/or.

In some embodiments, the product disclosed herein (which comprises glucosamine or a pharmaceutically acceptable salt thereof) and may comprise one or more additional agents, is administered together with a transdermal permeation enhancer. In further embodiments, the transdermal permeation enhancer promotes transdermal penetration of the glucosamine. In some embodiments, the transdermal permeation enhancer comprises one or more of: sea salt, urea, a sulphoxide, an azone, an oxazolidinone, a pyrrolidone, a fatty alcohol, a fatty acid ester, a fatty acid, a fatty alcohol ether, an enzyme, a surfactant, an essential oil, a terpene, a terpenoid, menthol, camphor, phenol, capsacin, or any other counter-irritant. In further embodiments, where a transdermal permeation enhancer comprises sea salts or dead sea salts, the salt includes one or more of: sodium chloride, magnesium chloride, potassium chloride, or magnesium sulfate.

In some embodiments, sea salts and/or dead sea salts create an osmotic gradient to facilitate, accelerate, and/or enhance absorption of glucosamine or a pharmaceutically acceptable salt thereof and/or another agent.

In certain embodiments, the products disclosed herein further comprises, by way of non-limiting examples, agents such as solvents, cosolvents, emulsifiers, fragrances, lubricants, gelling agents, thickeners, spreading agents, humectants, antioxidants, preservatives, cosmetic ingredients, essential oils, such as, for example, lavender, a counterirritant, Epson salt, (in fact, any suitable additional agents, additives, excipients, etc.) and combinations thereof.

Any suitable amount of any additional (other than glucosamine or a salt thereof) agent can be co-administered.

The product disclosed herein are suitable for the treatment of a wide range of conditions. These products can be used to treat a condition involving the outside of a subject's body, such as external bodily pain and/or discomfort. In certain embodiments, the condition is improved by such treatment and the improvement lasts after the treatment has been removed, for, for example, 72 hours, a week, or more.

### METHOD OF USE

The conditions that may be treated by a product disclosed herein include, for example, pain, inflammation, injury, arthritis, and osteoarthritis. The products disclosed herein are suitable for the treatment of a condition involving, by way of non-limiting examples, muscle pain, dental pain, period pain, back pain, neck pain, muscle injury, muscle strain, tendon injury, tendon strain, tendon inflammation, ligament injury, ligament strain, ligament inflammation, joint degradation, joint injury, joint wear and tear, joint inflammation, and/or joint pain, as well as pain, aches, and discomfort, caused by, for example, physical activity, sports, muscle fatigue, and gardening.

The products disclosed herein are suitable for the treatment of discomfort, such as aches, tiredness, etc.

A method is provided of reducing pain in a subject comprising immersing an area of the subject's body in pain in a topical fluid application apparatus disclosed herein, treating the area of the subject's body in pain with an article of manufacture disclosed herein, or using the kit disclosed herein to treat the subject.

A method is also provided of treating a condition disclosed herein in a subject in need of treatment comprising immersing an area of the subject's body in a topical fluid application apparatus disclosed herein, treating the area of the subject's body with an article of manufacture disclosed herein, or using the kit disclosed herein to treat the subject.

A method is provided of reducing discomfort, aches, stiffness, and stress in a subject comprising applying to an area of the subject's body in need of application a topical fluid application apparatus disclosed herein, treating the area of the subject's body in need thereof with an article of manufacture disclosed herein, or using a kit disclosed herein to treat the subject.

A method is provided of reducing discomfort, aches, stiffness, and stress in a subject comprising applying to an area of the subject's body in need of application a topical fluid application apparatus disclosed herein, treating the area of the subject's body disclosed herein with an article of manufacture disclosed herein, or using a kit disclosed herein to treat the subject.

A method is provided of reducing discomfort, aches, stiffness, and stress in a subject comprising applying to an area of the subject's body in need of application a topical fluid application apparatus disclosed herein, treating the area of the subject's body in pain with an article of manufacture disclosed herein, or using a kit disclosed herein to treat the subject.

A method is provided of relieving tired achy feet, hands, and joints in a subject comprising applying to an area of the subject's body in need of application a topical fluid application apparatus disclosed herein, treating the area of the subject's body in need of application with an article of manufacture disclosed herein, or using a kit disclosed herein to treat the subject.

A method is provided of providing soothing comfort to a subject comprising applying to an area of the subject's body in need of application a topical fluid application apparatus disclosed herein, treating the area of the subject's body in pain with an article of manufacture disclosed herein, or using a kit disclosed herein to treat the subject.

A method is provided of decreasing symptoms of arthritis in a subject comprising applying to an area of the subject's body in need of application a topical fluid application apparatus disclosed herein, treating the area of the subject's body in pain with an article of manufacture disclosed herein, or using a kit disclosed herein to treat the subject.

A method is provided of providing analgesia and anti-inflammatory properties to a subject comprising applying to an area of the subject's body in need of application a topical fluid application apparatus disclosed herein, treating the area of the subject's body in need of application with an article of manufacture disclosed herein, or using a kit disclosed herein to treat the subject.

In some embodiments, the products disclosed herein are suitable for the treatment of pain associated with one or more conditions, including, for example, osteoporosis. The pain includes, for example, joint pain, muscle pain, tendon pain, ligament pain, neck pain, lower back pain, hip pain, knee pain, ankle pain, foot pain, toe pain, shoulder pain, elbow pain, hand pain, and/or finger pain. The pain may be neck pain associated with one or more of cervical facet joint (zygapophyseal joint) disease/pain/arthritis, cervical discogenic pain, cervical radiculopathy, brachial plexopathy, neck ligament strain, and/or neck muscle strain. In further embodiments, lower back pain is associated with one or more of lumbar facet joint (zygapophyseal joint), disease/pain/arthritis, lumbar discogenic pain, sacroiliac joint pain, sciatica, lumbosacral radiculopathy, back ligament strain, and/or back muscle strain. In further embodiments, hip pain is associated with one or more of hip bursitis, iliotibial band pain, hip labral tear, hip impingement syndrome, and/or iliopsoas tendonitis. In further embodiments, knee pain is associated with one or more of knee meniscus tear, knee ligament strain, knee tendonitis, patellofemoral syndrome (chondromalacia patella), and/or osteochondritis dissecans. In further embodiments, ankle pain is associated with one or more of ankle sprain and/or osteochondritis dissecans. In further embodiments, foot pain is associated with one or more of metatarsal arthritis, metatarsalgia, plantar fasciitis, and/or calcaneus spur. In further embodiments, shoulder pain is associated with one or more of, shoulder impingement syndrome, rotator cuff tear, shoulder labrum tear, biceps tendonitis, and/or rotator cuff tendonitis. In further embodiments, elbow pain is associated with one or more of lateral epicondylitis, medial epicondylitis, and/or olecranon bursitis. In further embodiments, hand pain is associated with carpal tunnel syndrome.

In some embodiments, the products disclosed herein are suitable for the treatment of inflammation. In further embodiments, the products disclosed herein are suitable for the treatment of inflammation associated with one or more conditions described herein. For example, in various embodiments, the products disclosed herein are suitable for the treatment of joint inflammation, muscle inflammation, tendon inflammation, ligament inflammation, neck inflammation, lower back inflammation, hip inflammation, knee inflammation, ankle inflammation, foot inflammation, toe inflammation, shoulder inflammation, elbow inflammation, hand inflammation, and/or finger inflammation.

Osteoarthritis, an example of a joint condition, also known as degenerative arthritis or degenerative joint disease, is a group of mechanical abnormalities involving degradation of joints, including articular cartilage and subchondral bone. In some cases; symptoms include, for example, joint pain, tenderness, stiffness, locking, and the presence of increased intra-articular fluid.

Some studies have shown that oral administration of the monosaccharide amino sugar glucosamine, a constituent of hyaluronic acid, have mild to moderate efficacy in the treatment of joint conditions such as osteoarthritis. But oral glucosamine treatments have limitations because though glucosamine is well absorbed orally, it undergoes substantial first-pass metabolism. First-pass metabolism (also known as presystemic metabolism) is a phenomenon of drug metabolism whereby the concentration of a drug is greatly reduced as it passes through the liver before it reaches the systemic circulation. The first pass through the liver greatly reduces the bioavailability of glucosamine.

In some embodiments, the products disclosed herein are suitable for the treatment of arthritis. In further embodiments, the arthritis is psoriatic arthritis, juvenile idiopathic arthritis, septic arthritis, rheumatoid arthritis, or osteoarthritis. In various embodiments, the arthritis affects one or more of the neck, lower back, hip, knee, ankle, foot, toe, shoulder, elbow, hand, and/or finger. In a specific embodiment, the products, compositions, and methods described herein are suitable for the treatment of osteoarthritis of for example, the neck, lower back, hip, knee, ankle, foot, toe, shoulder, elbow, hand, and/or finger.

In some embodiments, the products disclosed herein are suitable for the treatment of injury to, by way of non-limiting examples, joints, muscles, tendons, and ligaments. In further embodiments, a joint injury is a knee meniscus tear. In some embodiments, the products disclosed herein are suitable for the prevention of joint, muscle, tension, and ligament injury.

In some embodiments, the products disclosed herein are suitable for the treatment of other conditions including, by way of non-limiting examples, fibromyalgia, lupus, tendonitis, bursitis, gout, pseudo-gout, cervical radiculopathy, lumbar radiculopathy, spondyloarthropathies, ankylosing spondylitis, chronic fatigue syndrome, complex regional pain syndrome, and/or cervicogenic headache.

In some embodiments, the products disclosed herein are suitable for the treatment of external body discomfort.

In some embodiments, the methods of treatment comprise administering to the subject glucosamine or a pharmaceutically acceptable salt thereof by immersion in a topical fluid application apparatus. Any suitable amount of times per day is contemplated. In some embodiments, administration is, by way of non-limiting examples 1, 2, 3, 4, 5, 6, 7, 8 or more times a day. In some embodiments, administration is more than 1, 2, 3, 4, 5, 6, 7, or 8 times a day. In some embodiments, administration is 1 to 5 times a day, 1 to 3 times a day, or 1 to 2 times a day. In a particular embodiment, administration is once a day.

Any suitable duration of treatment is contemplated.

In some embodiments, the methods of treatment further comprise administering to the subject oral agents. In further embodiments, the methods of treatment comprise orally administering to the subject glucosamine or a pharmaceutically acceptable salt thereof, chondroitin or a pharmaceutically acceptable salt thereof, and/or methylsulfonylmethane in combination therapy with one or more of the product disclosed herein.

### REFERENCE TO FIGURES

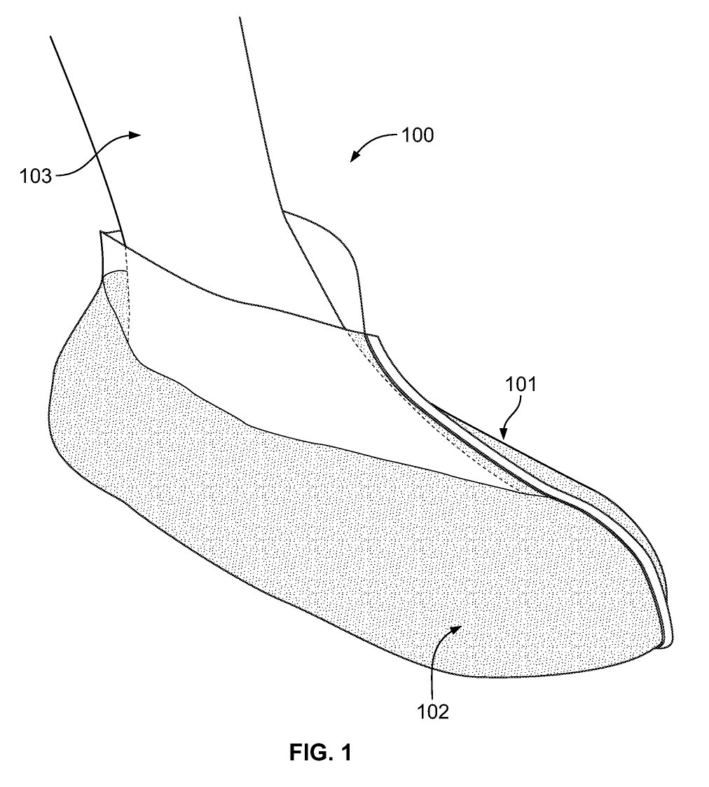

These and other embodiments of the invention are described below with reference to FIGS. 1-4 wherein like numerals are used throughout to denote like elements.

FIG. 1 illustrates an embodiment of a bootie 100, made of plastic, having an external portion 101 and an internal portion 102 that can hold a solution. A foot 103 can fit snugly into the bootie 100. The bootie can contain (1) solution; (2) a pad; or (3) dry or impregnated powder that would be solubilized by water or body fluids (e.g., sweat).

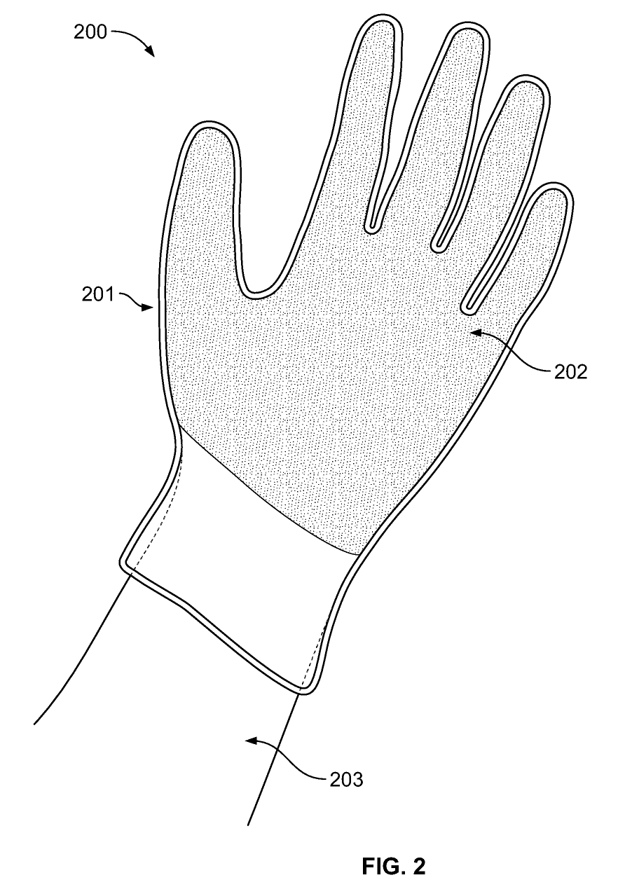

FIG. 2 illustrates an embodiment of a glove 200, made of plastic, having an external portion 201 and an internal portion 202 that can hold a solution. A hand 203 can fit snugly into the glove 200. The glove can contain (1) solution; (2) a pad; or (3) dry or impregnated powder that would be solubilized by water or body fluids (e.g., sweat).

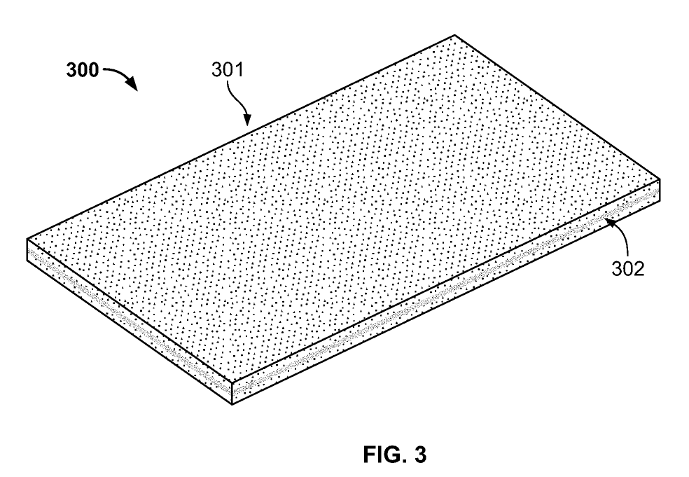

FIG. 3 illustrates an embodiment of a pliable sponge 300, having an external portion 301 and an internal portion 302 that can hold a solution. The sponge may optionally have an external plastic cover so as not to leak the solution. The sponge can be any size so as to accommodate a particular affected body part or site. The sponge can also take a custom shape to fit a given body part (e.g., shaped like a foot, a hand, a shoulder, or a knee).

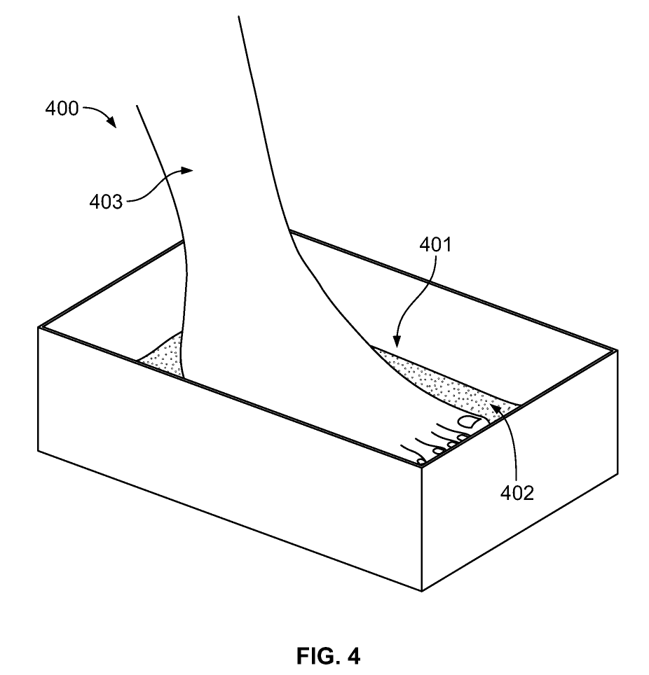

FIG. 4 illustrates an embodiment of a box 400, made of plastic, having an external portion 401 and an internal portion 402 that can hold a solution. A foot 403 can be immersed into the box 400.

### EXAMPLES

For this invention to be better understood, the following examples are set forth. These examples are for purposes of illustration only and are not be construed as limiting the scope of the invention in any manner.

### Example 1

Protocol Outline

Subjects: Normal healthy adults aged 40-75 with daily reported foot complaints (tired, achy, sore) that are not currently taking prescription or over the counter products for symptom relief. An attempt is made to recruit equal numbers of male and female participants.

Sample Size: 40 subjects; 20 per group

Treatment groups: Soothing foot bath topical fluid application apparatus (Glucosamine KCl), group A; Sham: Sucrose foot bath topical fluid application apparatus, group B.

Methods: Recruitment—Questionnaire evaluating health status and foot complaints; including visual analogue scale evaluation of current pain state.

Baseline—Questionnaire evaluating health status and foot complaints; including visual analogue scale evaluation of current pain state.

Treatment—Subjects are randomly assigned to one of two treatment groups and asked to submerge their right foot in a plastic box that contains 3 liters of room temperature tap water and 2 liters of boiling water plus one packet (50 grams) of the labeled “treatment pack” and stirred until in solution. Label is removed from pack and placed on chart. Foot remains submerged and stopwatch set for 1 hour. At the end of 1 hour, subject foot is dried and subject is asked to mark on the line (100 mm) the current state of their foot pain upon standing.

Evaluation—Subjects are asked to remain at the site and to mark the line to indicate their current pain status at 1 hour post treatment. They are then asked to complete a similar evaluation each hour for the next 6 hours and then each morning for the next 3 days. Reports should be returned to the site before compensation is provided. Upon return, subjects are to complete a final questionnaire that evaluates their current health status and has categorical ratings with respect to the ability of the product to relive their discomfort and a question regarding whether they would like to buy such product.

Adverse complaints are collected and analyzed.

Study personnel confirms that the subject has met the protocol requirements as an evaluable subject based on successful completion of the hour soaking and completion of the first assessment.

Statistics: Descriptive statistics is used to compare active to sham treatments. Primary endpoints are defined as differences at 4 hours post treatment. Secondary endpoints include differences immediately post treatment, 2 hours and 12, 24, and 48 hours post treatment. Adverse event reports are also be compared.

Missing data are imputed using the last observation carried forward approach.

Records: All reports are collected before compensation is provided and are maintained for a period of at least one year in hard copy form and 5 years electronically.

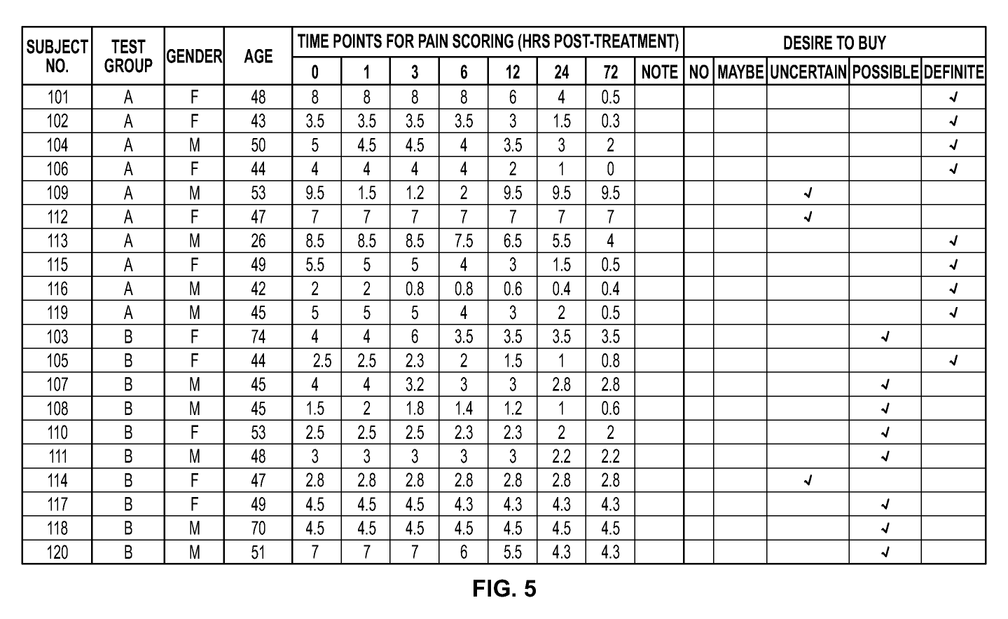

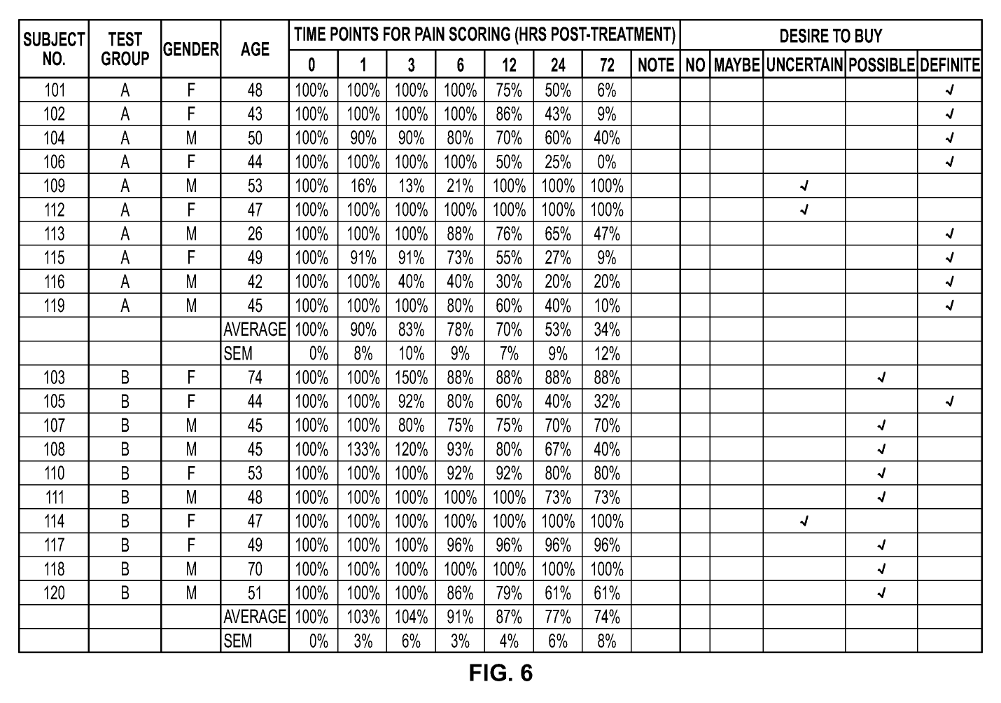

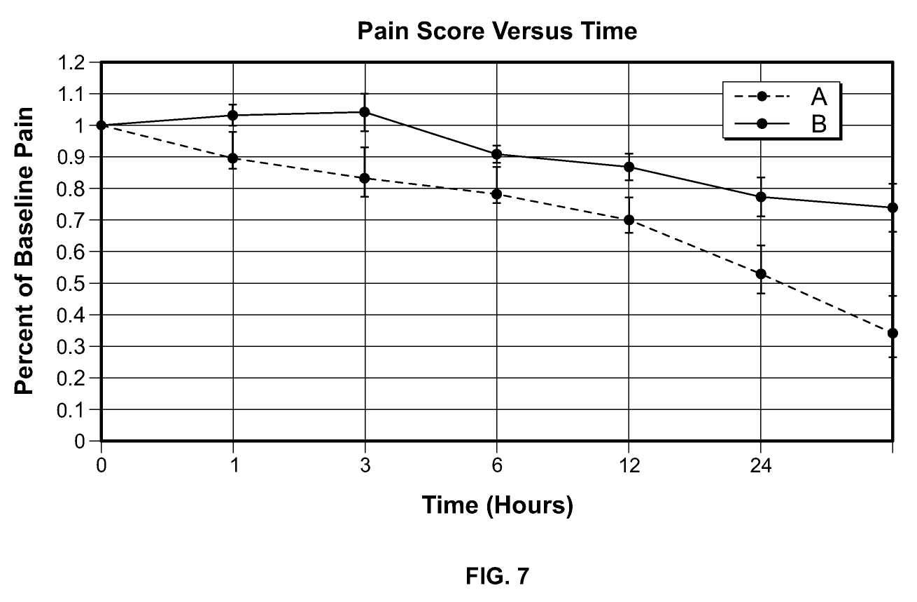

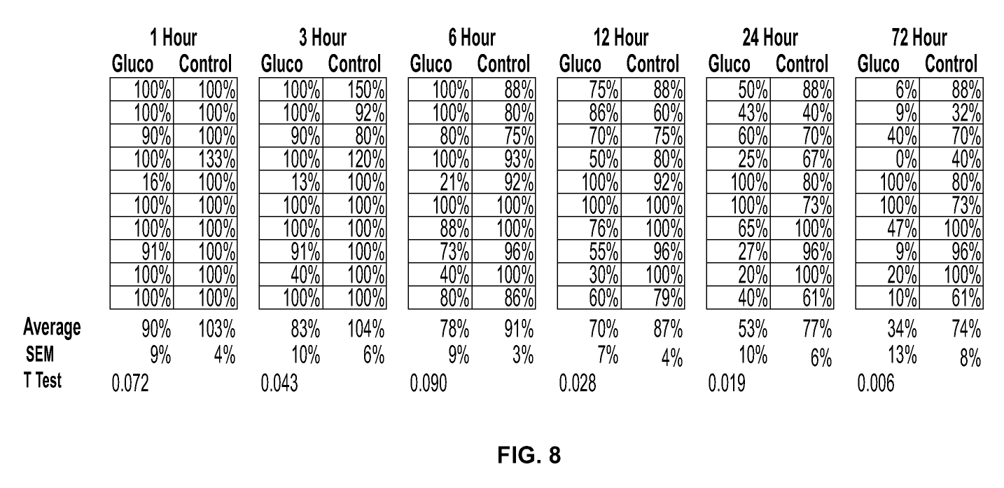

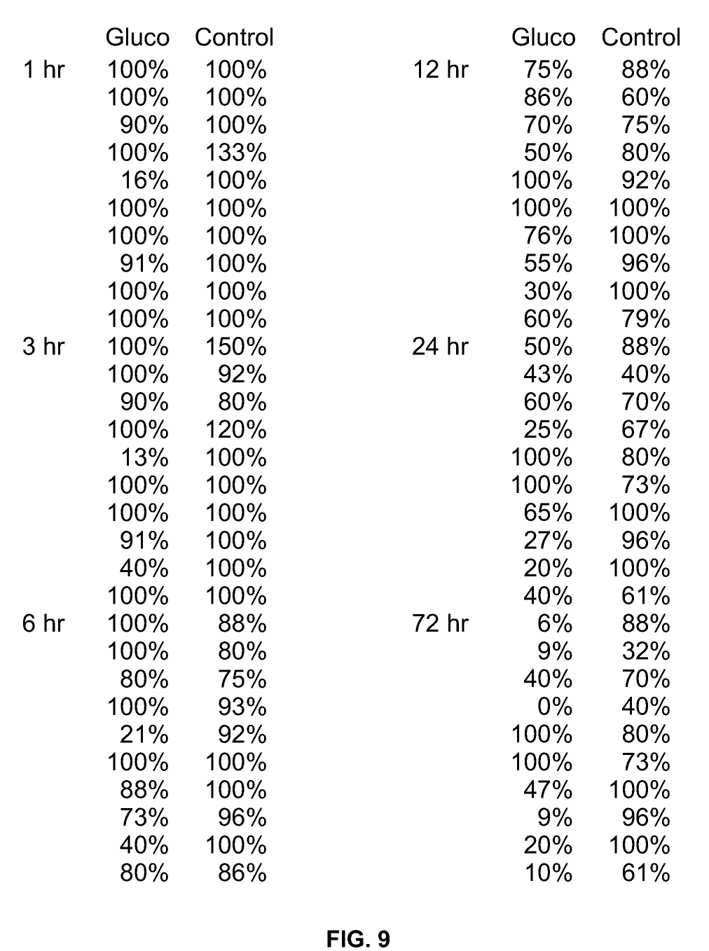

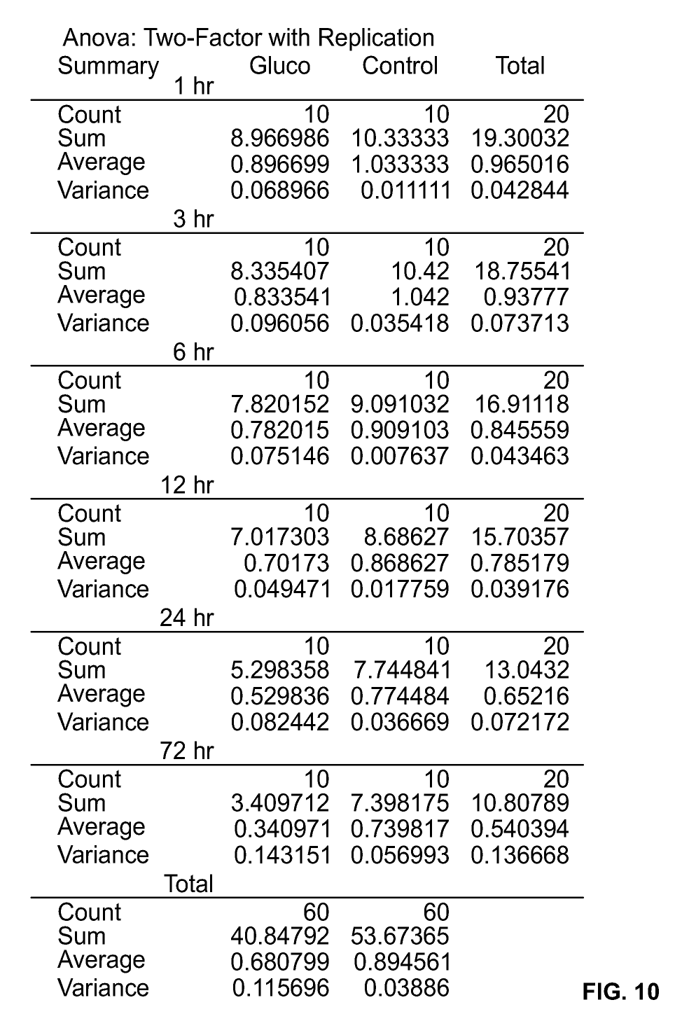

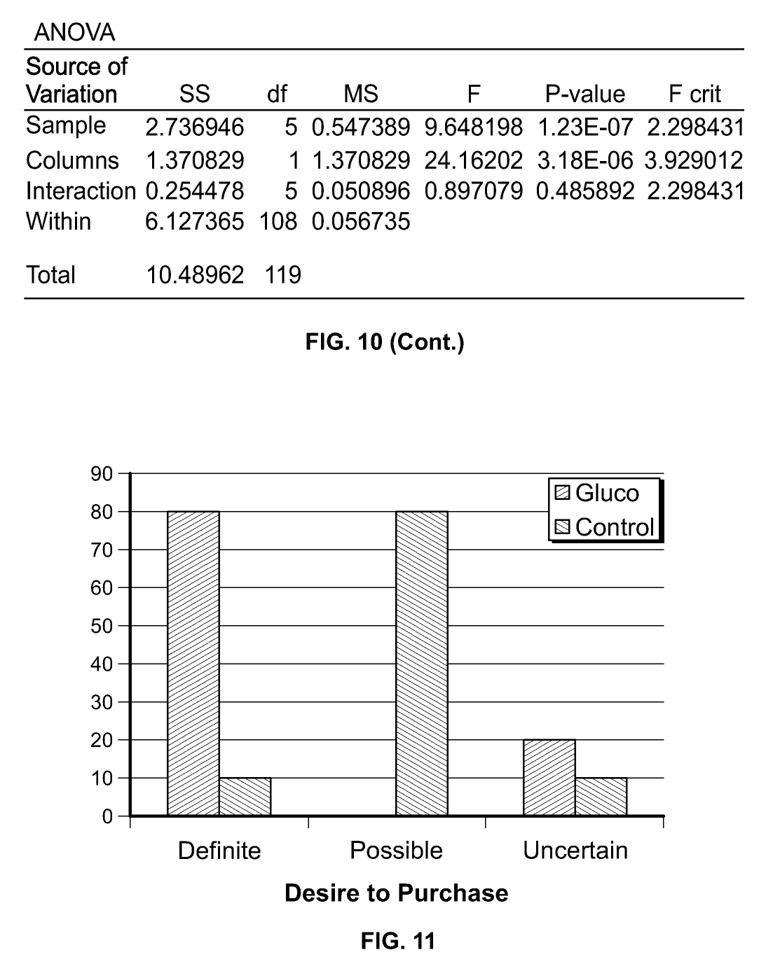

Results are shown in FIGS. 5-11. As can be seen, the group that received glucosamine (group A) rather than sucrose (group B) experienced sufficiently better pain relief; and that pain relief was long lasting—even 72 hours after treatment, many subjects treated with glucosamine experienced greater pain relief when compared to those who received sucrose.

### Other Embodiments

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A topical fluid application apparatus comprising glucosamine or a pharmaceutically acceptable salt thereof in a solution, wherein the topical fluid application apparatus is a flexible plastic form that fits over the hand or foot or is a box that fits over the foot of a subject, wherein the solution is added externally or is induced by occlusion of the subject's skin.

2. The topical fluid application apparatus of claim 1, wherein the flexible plastic form is a bootie.

3. The topical fluid application apparatus of claim 1, wherein the flexible plastic form is a glove.

4. The topical fluid application apparatus of claim 1, wherein the topical fluid application apparatus is a box that fits over the foot of a subject, wherein the solution is added externally or is induced by occlusion of the subject's skin.

5. The topical fluid application apparatus of claim 1, wherein the glucosamine or a pharmaceutically acceptable salt thereof is selected from: glucosamine, N-acetylglucosamine, glucosamine sulfate, glucosamine hydrochloride, glucosamine potassium sulfate, glucosamine hydrochloride, glucosamine potassium hydrochloride, glucosamine sulfate and combinations thereof.

6. The topical fluid application apparatus of claim 1, further comprising one or more of chondroitin sulfate and methyl sulfonylmethane.

7. The topical fluid application apparatus of claim 1, further comprising a transdermal permeation enhancer.

8. The topical fluid application apparatus of claim 1, further comprising one or more cosmetic ingredients and/or agents.

9. The topical application of claim 1, further comprising one or more of menthol, camphor, capsaicin, or any counter-irritant.

10. The topical application of claim 1, further comprising one or more additional agent.

11-18. (canceled)

19. An article of manufacture damp or impregnated with a solution comprising glucosamine or a pharmaceutically acceptable salt thereof, wherein said article of manufacture is a pliable sponge, a moist towelette, a pad, or an adhesive.

20. The article of manufacture of claim 19, wherein said article of manufacture is a pliable sponge.

21. The article of manufacture of claim 19, wherein the pliable sponge is covered by a plastic protective coating.

22. The article of manufacture of claim 19, wherein said article of manufacture is a moist towelette that is individually wrapped and impregnated with glucosamine solution.

23. The article of manufacture of claim 19, wherein said article of manufacture is a pad that is impregnated with the glucosamine solution.

24. The article of manufacture of claim 19, wherein the glucosamine or a pharmaceutically acceptable salt thereof is selected from: glucosamine, N-acetylglucosamine, glucosamine sulfate, glucosamine hydrochloride, and combinations thereof.

25. The article of manufacture of claim 19, further comprising one or more of chondroitin sulfate and methylsulfonylmethane.

26. The article of manufacture of claim 19, further comprising a transdermal permeation enhancer.

27. The article of manufacture of claim 19, further comprising one or more cosmetic ingredients and/or agents.

28. A method of reducing pain in a subject comprising applying to an area of the subject's body in pain a topical fluid application apparatus comprising glucosamine or a pharmaceutically acceptable salt thereof in a solution, wherein the topical fluid application apparatus is a flexible plastic form that fits over the hand or foot or is a box that fits over the foot of a subject, wherein the solution is added externally or is induced by occlusion of the subject's skin or treating the area of the subject's body in pain with an article of manufacture damp or impregnated with a solution comprising glucosamine or a pharmaceutically acceptable salt thereof, wherein said article of manufacture is a pliable sponge, a moist towelette, a pad, or an adhesive.

29-35. (canceled)

36. The method of claim 28, further comprising one or more of chondroitin sulfate and methylsulfonylmethane.

37. The method of claim 28, further comprising a transdermal permeation enhancer.

38. (canceled)

39. The method of claim 28, wherein the subject is a human subject.

40. (canceled)

41. The method of claim 28, wherein the glucosamine or a pharmaceutically acceptable salt thereof is selected from: glucosamine, N-acetylglucosamine, glucosamine sulfate, glucosamine hydrochloride, glucosamine potassium sulfate, glucosamine hydrochloride, glucosamine potassium hydrochloride, glucosamine sulfate and combinations thereof.
